(19) United States
(12) Patent Application Publication
(10) Pub. No.: US 2014/0240666 A1
(43) Pub. Date: Aug. 28, 2014

(54) OCULAR FUNDUS INFORMATION ACQUISITION DEVICE, METHOD AND PROGRAM

(71) Applicant: Sony Corporation, Tokyo (JP)

(72) Inventor: Tomoyuki Ootsuki, Kanagawa (JP)

(73) Assignee: Sony Corporation, Tokyo (JP)

(21) Appl. No.: 14/173,278

(22) Filed: Feb. 5, 2014

(30) Foreign Application Priority Data
Feb. 22, 2013 (JP) 2013-033495

(51) Int. Cl. A61B 3/00; A61B 3/14

(52) U.S. Cl. CPC A61B 3/0025; A61B 3/145; A61B 3/0091
USPC 351/206; 351/246

(57) ABSTRACT

An ocular fundus information acquisition device includes: a fixation target provision section configured to provide a continuously moving fixation target; an ocular fundus image acquisition section configured to acquire an image of an ocular fundus in a subject's eye while the subject is closely watching the continuously moving fixation target; and an ocular fundus information acquisition section configured to acquire ocular fundus information from the acquired ocular fundus image.

### CROSS REFERENCE TO RELATED APPLICATIONS

This application claims the benefit of Japanese Priority Patent Application JP 2013-033495 filed Feb. 22, 2013, the entire contents of which are incorporated herein by reference.

### BACKGROUND

The present technology relates to an ocular fundus information acquisition device, method and program, and more specifically to an ocular fundus information acquisition device, method and program that are capable of acquiring high-quality information on an ocular fundus.

In diagnosing certain diseases of an ocular fundus, there are cases where information regarding the ocular fundus is necessary. In addition detailed information on an ocular fundus facilitates an accurate and immediate diagnosis.

For example, when a photograph of an ocular fundus is taken in a single shot, the field of view of the acquired image is typically limited. So it may not be wide enough to diagnose the condition of the ocular fundus. In order to acquire an ocular fundus image with a wide field of view, a method of capturing multiple still images of an ocular fundus and piecing these images together has been widely employed.

For example, Japanese Unexamined Patent Application Publication No. 2004-254907 proposes a method of acquiring an ocular fundus image with a wide field of view by storing different sites in advance and sequentially photographing a fixation target while moving the fixation target to these sites in the stored order.

### SUMMARY

The above-described method acquires an image with a wide field of view by piecing multiple images together so that overlapping regions therebetween are small. As a result the borders between the adjacent images may become noticeable and the quality of the acquired image is deteriorated.

FIG. 1 illustrates an exemplary ocular fundus image with a wide field of view. The ocular fundus image in FIG. 1 contains an optic papilla 1, a macular area 2, and blood vessels 3. In order to create this image, still images acquired in ten shots are pieced together. A border 4 is present between the adjacent still images, and defines a region corresponding to a single frame image. An image 5-i (i=1 to 10) that contains a certain area of a single frame image and an adjacent image 5-j (j=1 to 10≠i) that contains a certain area of another single image are pieced together so that an overlapping region therebetween is small.

FIG. 2 is an explanatory, schematic view of a method of piecing images together. As illustrated in FIG. 2, the image 5-1 contains a certain area of a single frame image, and the image 5-2 contains a certain area of another single frame. Further the images 5-1 and 5-2 are pieced together such that the region in the image 5-1 on the left side of a left dotted line is used. Moreover the image 5-2 and the image 5-3 that contains a certain area of still another single frame image are pieced together such that the region in the image 5-2 on the left side of a right dotted line is used. The image 5-3 and another image are also pieced together likewise. In the resultant image created in this manner, the borders 4 may be noticeable between the adjacent images because of the difference in pixel values.

FIG. 3 is an explanatory view illustrating an exemplary arrangement of fixation targets 11-1 to 11-3. In the example of FIG. 3, three fixation targets 11-1 to 11-3, each of which is configured with a light emitting diode (LED), are lighted at different timings. For example, the above image 5-1 is acquired as a result of photographing a subject that is watching the fixation target 11-1 closely. Likewise the image 5-2 is acquired as a result of photographing the subject that is watching the fixation target 11-2 closely; the image 5-3 is acquired as a result of photographing the subject that is watching the fixation target 11-3 closely.

When all of the images 5-1 to 5-3 acquired in the above manner are pieced together through their circumferences, the resultant image may exhibit low viewability, because the pixel values in the vicinity of each border 4 differ from one another, as illustrated in FIG. 1.

It is desirable to provide an ocular fundus information acquisition device, method and program that are capable of acquiring high-quality information on an ocular fundus.

An ocular fundus information acquisition device according to an embodiment of the present technology includes: a fixation target provision section configured to provide a continuously moving fixation target; an ocular fundus image acquisition section configured to acquire an image of an ocular fundus in a subject's eye while the subject is closely watching the continuously moving fixation target; and an ocular fundus information acquisition section configured to acquire ocular fundus information from the acquired ocular fundus image.

The ocular fundus image acquisition section may acquire a moving image of the ocular fundus.

The fixation target provision section may provide a blinking internal fixation target.

The ocular fundus information acquisition section may select, as a target image, a frame image in the moving image which has been acquired during a period in which the fixation target is not lighted, and the ocular fundus information is acquired from the selected target image.

An ocular fundus image provision section configured to provide the image of the ocular fundus in the subject's eye which has been acquired while the subject is closely watching the continuously moving fixation target may be further provided.

The ocular fundus image provision section may provide the ocular fundus image during a period in which the fixation target is not lighted, the ocular fundus image being the frame image in the moving image, and may provide the ocular fundus image during a period in which the fixation target is lighted, the ocular fundus image being the frame image in the moving image which has been acquired during the period in which the fixation target is not lighted.

The ocular fundus information acquisition section may acquire the ocular fundus image with a wide field of view.

The ocular fundus information acquisition section may acquire the ocular fundus image with super resolution.

The ocular fundus information acquisition section may acquire a 3D shape of the ocular fundus.

The ocular fundus information acquisition section may acquire a 3D ocular fundus image.

The ocular fundus image acquisition section may acquire the moving image of the ocular fundus with infrared light and a still image of the ocular fundus with visible light. The ocular fundus information acquisition section may acquire a 3D shape of the ocular fundus from the infrared light moving image of the ocular fundus, and acquire a visible light 3D ocular fundus image by mapping the visible light still image onto the 3D shape while adjusting a location of the visible light still image with respect to the 3D shape.

According to an embodiment of the present technology, a fixation target provision section provides a continuously moving fixation target; an ocular fundus image acquisition section acquires an image of an ocular fundus in a subject's eye while the subject is closely watching the continuously moving fixation target; and an ocular fundus information acquisition section acquires ocular fundus information from the acquired ocular fundus image.

A method and program according to an embodiment of the present technology are a method and program, respectively, that correspond to the above ocular fundus information acquisition device according to an embodiment of the present technology.

An embodiment of the present technology, as described above, successfully provides an ocular fundus information acquisition device, method and program that are capable of acquiring high-quality information on an ocular fundus.

### BRIEF DESCRIPTION OF THE DRAWINGS

FIG. 1 illustrates an exemplary ocular fundus image with a wide field of view.

FIG. 2 is an explanatory, schematic view of a method of piecing images together.

FIG. 3 is an explanatory view illustrating an exemplary arrangement of fixation targets.

FIG. 4 is a block diagram illustrating an exemplary configuration of an ocular fundus information acquisition device according to an embodiment of the present technology.

FIG. 5 is a block diagram illustrating an exemplary functional configuration of the ocular fundus information acquisition section.

FIG. 6 illustrates an exemplary configuration of the fixation target provision section.

FIGS. 7A and 7B are timing charts of frame images, which is used to explain a method of selecting the frame images.

FIG. 8 illustrates an exemplary outer configuration of the ocular fundus information acquisition device.

FIGS. 9A and 9B are explanatory views of the movement of the fixation target.

FIG. 10 is an explanatory view of a change in the ocular fundus image.

FIG. 11 is a flowchart of processing of acquiring a wide-field ocular fundus image.

FIG. 12 illustrates an exemplary wide-field ocular fundus image.

FIG. 13 is an explanatory view of a method of synthesizing images.

FIGS. 14A and 14B are explanatory, schematic views of the method of synthesizing images.

FIGS. 15A and 15B are explanatory views of the movement of the fixation target.

FIG. 16 is a flowchart of processing of acquiring a super-resolution ocular fundus image.

FIG. 17 is a block diagram illustrating an exemplary functional configuration of an ocular fundus information acquisition section.

FIG. 18 is a flowchart of processing of generating a super-resolution ocular fundus image.

FIGS. 19A and 19B are explanatory views of the movement of the fixation target.

FIG. 20 is a flowchart of processing of acquiring the 3D shape of the ocular fundus.

FIG. 21 illustrates a cross section of an exemplary 3D shape of the ocular fundus.

FIG. 22 is a flowchart of processing of acquiring a 3D ocular fundus image.

FIG. 23 illustrates an exemplary 3D ocular fundus image.

FIG. 24 is a block diagram illustrating an exemplary configuration of an ocular fundus information acquisition device.

FIG. 25 is a flowchart illustrating processing of providing a captured image.

FIGS. 26A and 26B are explanatory views of an image capturing element that captures a moving image with infrared light and a still image with visible light.

FIG. 27 is an explanatory view of a method of capturing a moving image with infrared light and a still image with visible light.

FIG. 28 is a flowchart illustrating processing of acquiring a 3D ocular fundus image.

### DETAILED DESCRIPTION OF EMBODIMENTS

Hereinafter some embodiments of the present technology will be described in the following order.

[First Embodiment: Acquisition of Ocular Fundus Image with Wide-field of View]
1. Configuration of ocular fundus information acquisition device
2. Fixation target provision section
3. Processing of acquiring wide-field ocular fundus image
[Second Embodiment: Acquiring Ocular Fundus Image with Super Resolution]
4. Ocular fundus image with super-resolution
5. Another configuration of ocular fundus information acquisition section
6. Configuration of super-resolution processing section
7. Processing of generating ocular fundus image

### [Third Embodiment: Acquiring 3D Shape]

8. Acquiring 3D shape

### [Fourth Embodiment: Acquiring 3D Ocular Fundus Image]

9. 3D ocular fundus image
[Fifth Embodiment: Configuration with Ocular Fundus Image Provision Section]
10. Another configuration of ocular fundus information acquisition device
[Sixth Embodiment: Acquiring Moving Image with Infrared Light]
11. Acquiring moving image with infrared light and still image with visible light
12. Application of the present technology to program
13. Other configurations

### First Embodiment

### Acquisition of Ocular Fundus Image with Wide-field of View

### (Configuration of Ocular Fundus Information Acquisition Device)

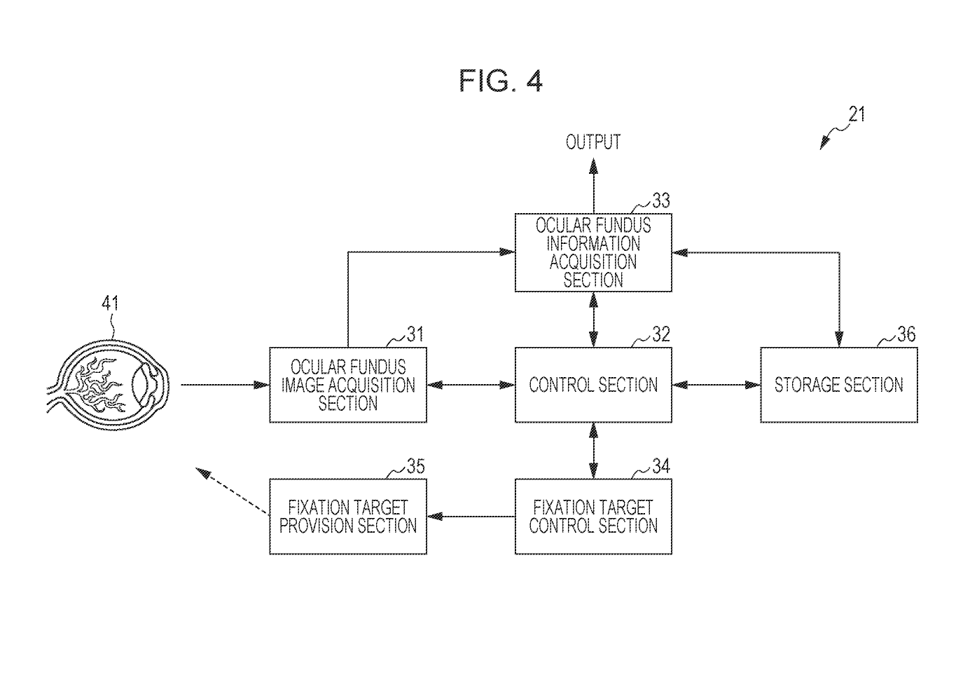

FIG. 4 is a block diagram illustrating an exemplary configuration of an ocular fundus information acquisition device 21 according to a first embodiment of the present technology. The ocular fundus information acquisition device 21 includes an ocular fundus image acquisition section 31, a control section 32, an ocular fundus information acquisition section 33, a fixation target control section 34, a fixation target provision section 35, and a storage section 36.

The ocular fundus image acquisition section 31 has, for example, a charge coupled device (CCD) or complementary metal oxide semiconductor (CMOS) image sensor, and captures an image of the ocular fundus in a subject's eye 41 to be examined. The control section 32 is configured with, for example, a central processing unit (CPU), and controls the operations of the ocular fundus image acquisition section 31, the ocular fundus information acquisition section 33, the fixation target control section 34, and the like. The ocular fundus information acquisition section 33 is configured with, for example, a digital signal processor (DSP), and acquires ocular fundus information to output it to a recording section (not illustrated) or the like.

The fixation target control section 34 controls the operation of the fixation target provision section 35 under the control of the control section 32. The fixation target provision section 35 provides a fixation target for the subject. The fixation target guides the eyepoint of the subject's eye 41 in order to acquire an image of a predetermined part of the ocular fundus. The storage section 36 stores programs, data and the like to be handled by the control section 32 and the ocular fundus information acquisition section 33.

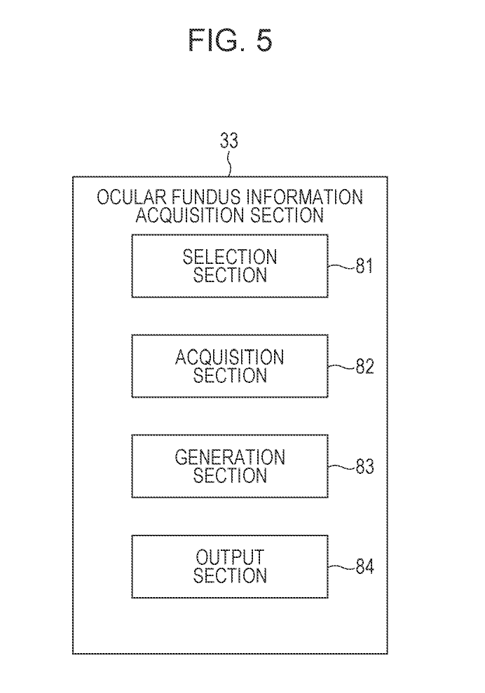

FIG. 5 is a block diagram illustrating an exemplary functional configuration of the ocular fundus information acquisition section 33. The ocular fundus information acquisition section 33 includes a selection section 81, an acquisition section 82, a generation section 83, and an output section 84.

The selection section 81 acquires process target frame images from frame images that make up a moving image supplied from the ocular fundus image acquisition section 31. The acquisition section 82 acquires a 3D shape and the like of the ocular fundus on the basis of a positional relationship among the ocular fundi in the process target frame images. The generation section 83 generates ocular fundus information, including a wide-field ocular fundus image, a super-resolution ocular fundus image, a 3D shape, and a 3D ocular fundus image. The output section 84 outputs the generated ocular fundus information.

### (Fixation Target Provision Section)

The fixation target provision section 35 in the first embodiment provides a fixation target that is continuously moving over a predetermined range. The fixation target may be a bright point on a liquid crystal display, an organic electro luminescence (EL) display, or some other display.

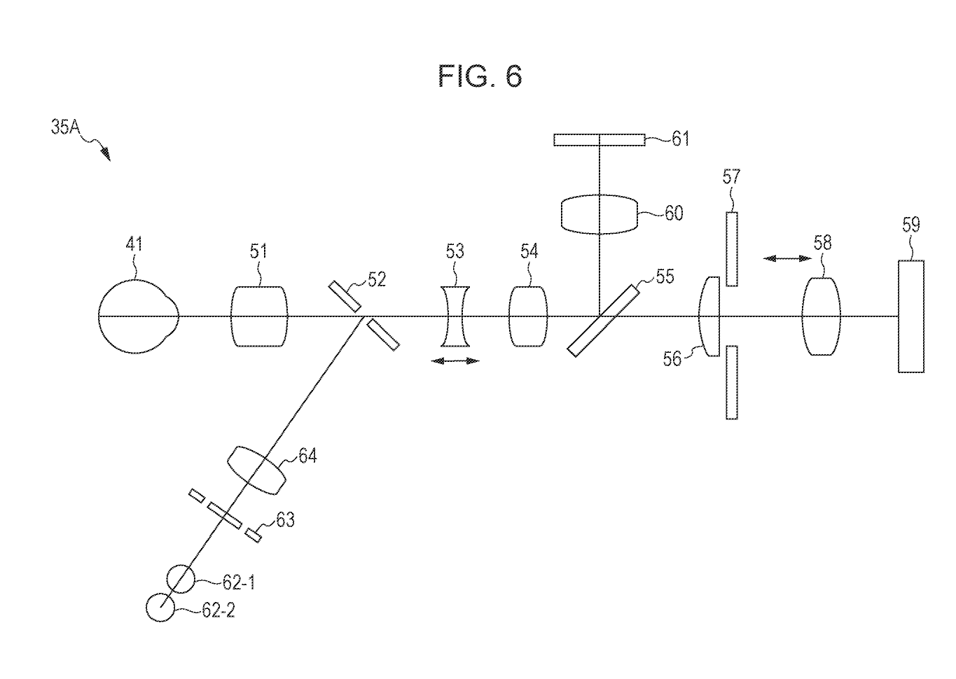

The fixation target in the first embodiment may be either an internal or external fixation target. FIG. 6 illustrates an exemplary configuration of a fixation target provision section 35A that provides an internal fixation target. Specifically some of the components in FIG. 6 constitute the ocular fundus image acquisition section 31.

The exemplary overall optical system in FIG. 6 includes an illumination optical system, a photographic optical system, and a fixation target optical system.

The components of the illumination optical system are a visible light source 62-1, an infrared light source 62-2, a ring diaphragm 63, a lens 64, a perforated mirror 52, and an objective lens 51. Here the visible light source 62-1 generates visible light and the infrared light source 62-2 generates infrared light; and either one of them is used as appropriate. The components of the photographic optical system are the objective lens 51, the perforated mirror 52, a focus lens 53, a photographic lens 54, a half mirror 55, a field lens 56, a field diaphragm 57, an imaging lens 58, and an image capturing element 59. The components of the fixation target optical system are a fixation target provision element 61, an imaging lens 60, the half mirror 55, the photographic lens 54, the focus lens 53, the perforated mirror 52, and the objective lens 51.

The fixation target provision element 61 is configured with, for example, a liquid crystal display, an organic EL display, or some other display that is capable of showing a continuously moving bright point. The image of the bright point disposed at any given site in the fixation target provision element 61 is supplied to the subject's eye 41 through the imaging lens 60, the half mirror 55, the photographic lens 54, the focus lens 53, the perforated mirror 52, and the objective lens 51, so that it is observed as the fixation target by the subject's eye 41.

When the visible light source 62-1 emits visible light or the infrared light source 62-2 emits infrared light, the visible or infrared light is incident on the perforated mirror 52 through the ring diaphragm 63 and the lens 64. Then, the incident light is reflected by the perforated mirror 52, and shines on the subject's eye 41 through the objective lens 51.

The light reflected by the subject's eye 41 enters the image capturing element 59 through the objective lens 51, a through-hole in the perforated mirror 52, the focus lens 53, the photographic lens 54, the half mirror 55, the field lens 56, the field diaphragm 57, and the imaging lens 58.

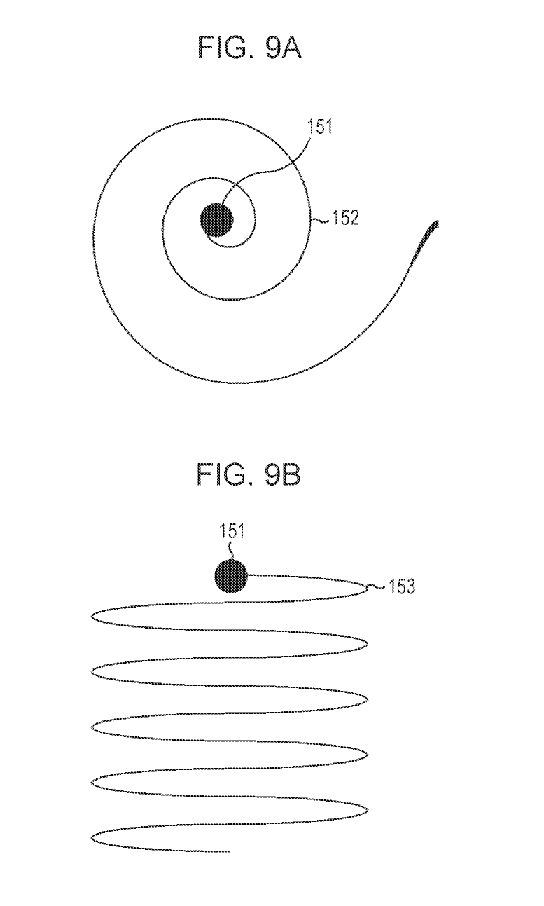

When the subject watches the fixation target closely, the subject's eye 41 follows the movement of the fixation target (a moving fixation target 151 in FIG. 9 which will be described later) in the fixation target provision element 61. It is thus possible to move the subject's eye 41 to a desired site by changing the location of the fixation target as appropriate. This is how the image capturing element 59 captures an image of a desired region of the ocular fundus in the subject's eye 41.

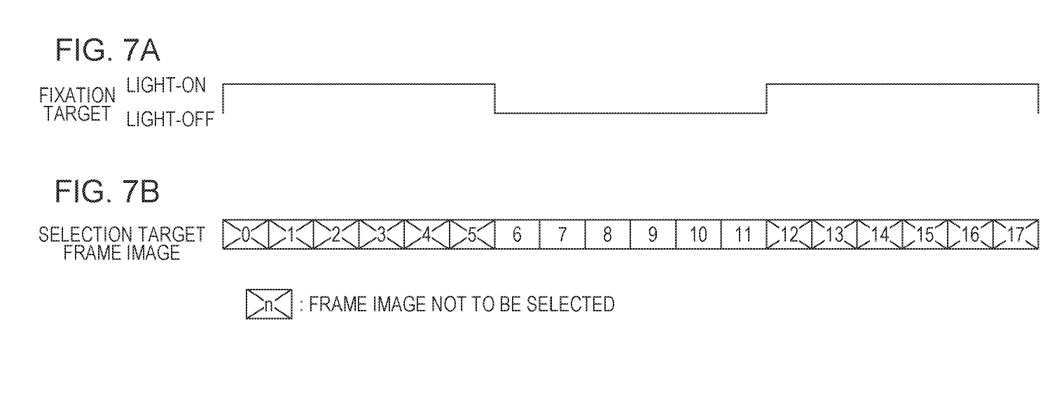

FIGS. 7A and 7B are timing charts of frame images, which is used to explain a method of selecting the frame images. When the photograph of the ocular fundus is taken while the fixation target appears in the subject's eye 41, a deteriorated image of the ocular fundus may be acquired, because the light from the fixation target is reflected by the subject's eye 41. Accordingly it is preferable that the fixation target blink, for example, as illustrated in FIGS. 7A and 7B. In the example of FIGS. 7A and 7B, the fixation target is lighted at the timings of frame images 0 to 5 and 12 to 17 out of the sequential frame images making up the moving image, in order to guide the subject's eye 41 to a predetermined site. In addition the fixation target is not lighted at the timing of frame images 6 to 11. Thus the fixation target continuously blinks in a period of capturing the twelve frame images.

Only the frame images (the frame images 6 to 11 in the example of FIGS. 7A and 7B) captured while the fixation target is not lighted are acquired as ocular fundus images. In other words the frame images (the frames 0 to 5 and 12 to 17 in the example of FIGS. 7A and 7B) captured while the fixation target is being lighted are not used.

For example, if the ocular fundus information acquisition device 21 employs the National Television System Committee (NTSC) scheme, its frame rate is 30 fps. In the case where the fixation target blinks in synchronization with this frame rate, the fixation target is lighted for (6×3/30) seconds and stops being lighted for (6×2/30) seconds. Alternatively the fixation target may be lighted for (6×2/30) seconds and stop being lighted for (6×3/30) seconds.

In the former case, the fixation target is lighted three times and stops being lighted twice in a second. Twelve frame images are thus acquired during the capturing of the moving image in a second. In the latter case, the fixation target is lighted twice and stops being lighted three times in a second. Eighteen images are thus acquired during the capturing of the moving image in a second.

Assuming that the blinking period synchronizes with a period of capturing ten frames, the fixation target is lighted for 5×3/30 seconds and stop being lighted for 5×3/30 seconds. In the latter case, the fixation target is lighted three times and stops being lighted three times. Fifteen frame images are thus acquired during the capturing of the moving image in a second.

Since the period during which the fixation target is not lighted becomes short as described above, the subject perceives the fixation target as continuously moving. This prevents the subject from misunderstanding that guidance of the subject's eye 41 has finished and returning the subject's eye 41 to the initial location. Consequently it is possible to capture the images of sequential parts of the ocular fundus in the subject's eye 41 by interpolating parts of the ocular fundus which correspond to the non-lighting periods preceding and following each the lighting period.

When the subject sequentially watches the fixation targets 11-1 to 11-3 arranged so as to be separated from one another as illustrated in FIG. 3, the ocular fundus images may be captured individually. Specifically, for example, the subject watches the lighted fixation target 11-1, and then after the subject's eye 41 stops moving, the still image of the ocular fundus is captured. After the image has been captured using the fixation target 11-1, the fixation target 11-2 is lighted in the wake of the fixation target 11-1. The subject watches the fixation target 11-2 closely, and then after the subject's eye 41 stops moving, the still image of the ocular fundus is captured. In this manner, an operation of capturing the still image of the ocular fundus is performed every time any of the fixation targets 11-1 to 11-3 is lighted. In this case, however, the subject is forced to repeatedly interrupt and resume watching it every time a lighted one of the fixation targets 11-1 to 11-3 is changed. This may make the subject feel inconvenienced.

In contrast when the fixation target 151 is continuously provided as in the first embodiment, it is only necessary for the subject to continuously follow the movement of the fixation target 151 with the subject's eye 41 without consideration of the capturing timing. Consequently the inconvenience for the subject is reduced in comparison with the case where the fixation targets 11-1 to 11-3 are arranged so as to be separated from one another, namely, the fixation target is provided intermittently as illustrated in FIG. 3.

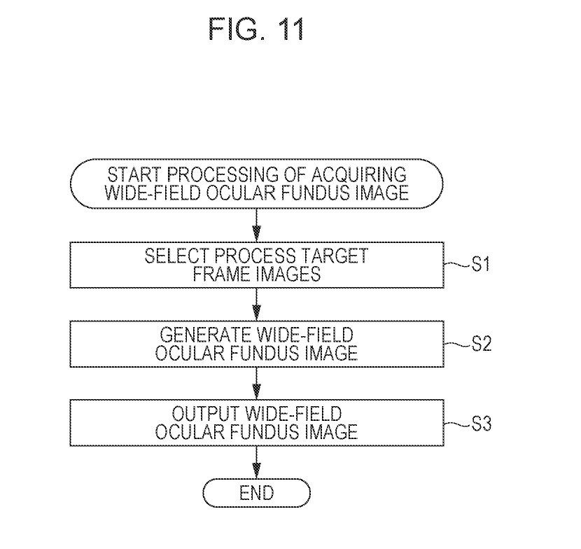

In a process of selecting a process target frame image at Step S1 or the like in FIG. 11 which will be described later, any given frame images may be selected from frame images acquired during the non-lighting period. Specifically either all the frame images or an arbitrary number of frame images may be selected from the frame images captured during the non-lighting period.

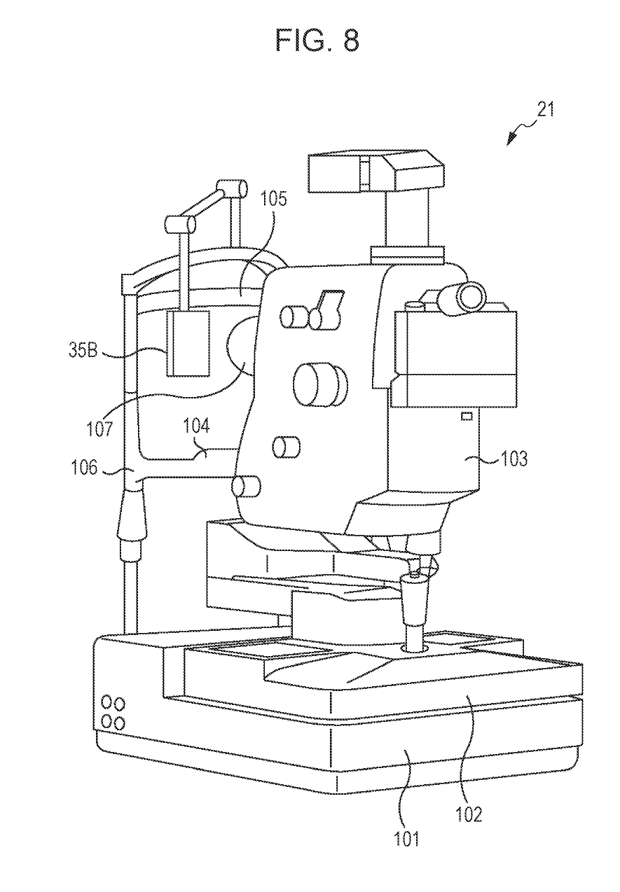

FIG. 8 illustrates an exemplary outer configuration of the ocular fundus information acquisition device 21 having a fixation target provision section 35B that provides an external fixation target. In the ocular fundus information acquisition device 21, a stand 102 is set on a base 101, and a main body 103 is installed on the stand 102. A supporting column 106 is disposed opposite the front of the main body 103. The supporting column 106 is provided with a forehead support 105 and a chin support 104. When the subject sets his or her forehead and chin on the forehead support 105 and the chin support 104, respectively, the ocular fundus information acquisition device 21 gets ready to capture an image of the ocular fundus through a photographic lens contained in a lens-barrel 107 of the main body 103. The main body 103 houses illumination and photographic optical systems, similar to the fixation target provision section 35A, as illustrated in FIG. 6, that provides the internal fixation target.

The supporting column 106 is equipped with a fixation target provision section 35B. The fixation target provision section 35B may be positioned on either side of the lens-barrel 107. The subject closely watches the fixation target on a display (not illustrated), as a fixation target provision element in the fixation target provision section 35B, with his or her eye that will not become a photographic target. When the eye watching the fixation target moves in response to the movement of the fixation target, the other eye (the subject's eye 41) also moves in the same direction, because the two human eyes move in synchronization with each other. This is how the subject's eye 41 is moved to and is positioned at a desired site.

In the case where the external fixation target is used as illustrated in FIG. 8, the process of selecting frames as in FIGS. 7A and 7B may not be necessary, because no fixation target appears in the subject's eye 41.

The fixation target provision element in the fixation target provision section 35B is configured with a liquid crystal display, an organic EL display, or some other display, similar to the fixation target provision element 61 in FIG. 6. However any given element capable of providing a continuously moving fixation target may be used as the fixation target provision element in the fixation target provision section 35B or the fixation target provision element 61 in FIG. 6. Instead of such a display, a mechanism that is capable of continuously moving a fixation target composed of a light-emitting unit such as a light emitting diode (LED) may be provided.

A description will be given below of an overall operation of the ocular fundus information acquisition device 21 in FIG. 4. The fixation target control section 34 controls the fixation target provision section 35 in such a way that the fixation target continuously moves so as to trace a predetermined locus, as illustrated in FIG. 9A or 9B. Meanwhile the ocular fundus image acquisition section 31 captures a moving image of the ocular fundus while the subject's eye 41 is being guided by the fixation target.

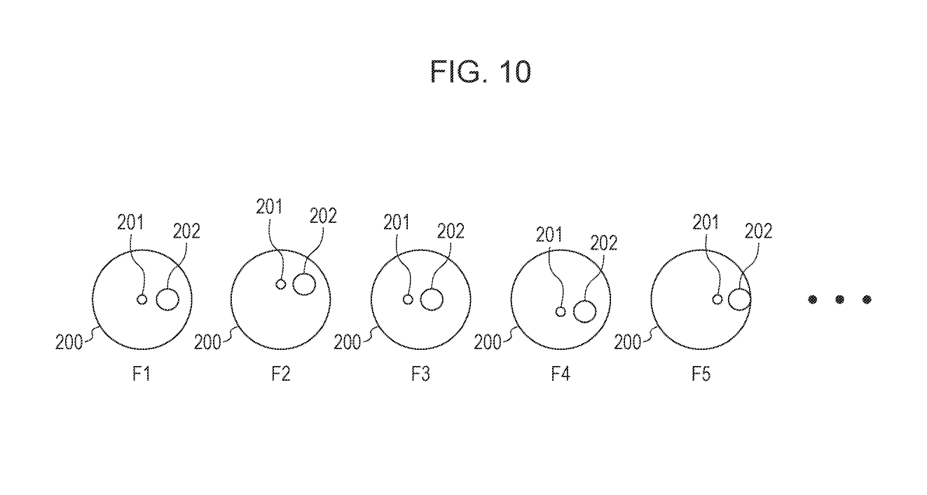

FIGS. 9A and 9B are explanatory views of the movement of a fixation target when an ocular fundus image with a wide field of view is captured; FIG. 10 is an explanatory view of a change in the ocular fundus image. In the example of FIG. 9A, a fixation target 151 continuously moves from the inner side toward the outer side so as to trace a spiral locus 152. In the example of FIG. 9B, the fixation target 151 continuously moves so as to trace a sinusoidal locus 153.

When the fixation target 151 continuously moves from the inner side toward the outer side so as to trace the spiral locus 152, for example, as illustrated in FIG. 9A, a captured ocular fundus image 200 changes as in FIG. 10. In FIG. 10, ocular fundus images 200 of subsequent frames F1, F2, F3 and so on making up a moving image are illustrated. In the ocular fundus images 200 of the subsequent frames F1, F2, F3 and so on, the locations of a macular area 201 and an optic papilla 202 sequentially move upward or downward while gradually moving outward. This moving image is acquired by the ocular fundus image acquisition section 31, and is supplied to the ocular fundus information acquisition section 33. It is to be noted that FIG. 10 simply shows the principal of the change in a captured ocular fundus image, and the actual locations of the macular area 201 and the optic papilla 202 do not change so greatly.

The ocular fundus information acquisition section 33 acquires desired ocular fundus information on the basis of the moving image captured by the ocular fundus image acquisition section 31, and outputs it. The ocular fundus information is output to the storage section 36 and stored therein, or to a monitor (not illustrated) and displayed thereon. The control section 32 controls the entire device in such a way that the series of operations are performed in conjunction with one another.

Next a description will be given of a process through which the ocular fundus information acquisition section 33 acquires a wide-field ocular fundus image, a super-resolution ocular fundus image, a 3D shape, and a 3D ocular fundus image, as the ocular fundus information.

### (Process of Acquiring Wide-Field Ocular Fundus Image)

FIG. 11 is a flowchart of processing of acquiring a wide-field ocular fundus image which is performed by the ocular fundus information acquisition section 33. At Step S1, the selection section 81 selects process target frame images from frame images that make up a moving image received from the ocular fundus image acquisition section 31. This selection process may be performed as necessary. For example, in the case where the internal fixation target is provided using the visible light source 62-1, the process target frame images may be selected in accordance with the above timing chart in FIGS. 7A and 7B. Specifically image frames captured during the period in which the fixation target 151 is not lighted may be selected from the sequential frame images, as the process target frame images.

In the case where the external fixation target is used as illustrated in FIG. 8, the selection process may be skipped in order to use all the frame images. Even in the case where the internal fixation target is used as illustrated in FIG. 6, the selection process may also be skipped under the condition that the infrared light source 62-2 is used, an element that is capable of receiving infrared light is used as the image capturing element 59, and an infrared light transmission filter (i.e. visible light cut filter) is set in front of the image capturing element 59.

At Step S2, the generation section 83 generates a wide-field ocular fundus image. In more detail the generation section 83 adjusts the relative position of the process target frame images selected in the process at Step S1. If the same part of the ocular fundus is contained in multiple images, the corresponding pixel values of these images are weighted and added (e.g. averaged). As a result a wide-field ocular fundus image is generated. At Step S3, the output section 84 outputs the wide-field ocular fundus image generated through the process at Step S2. This resultant panoramic image is supplied to a display viewed by a doctor or is stored in the recording section.

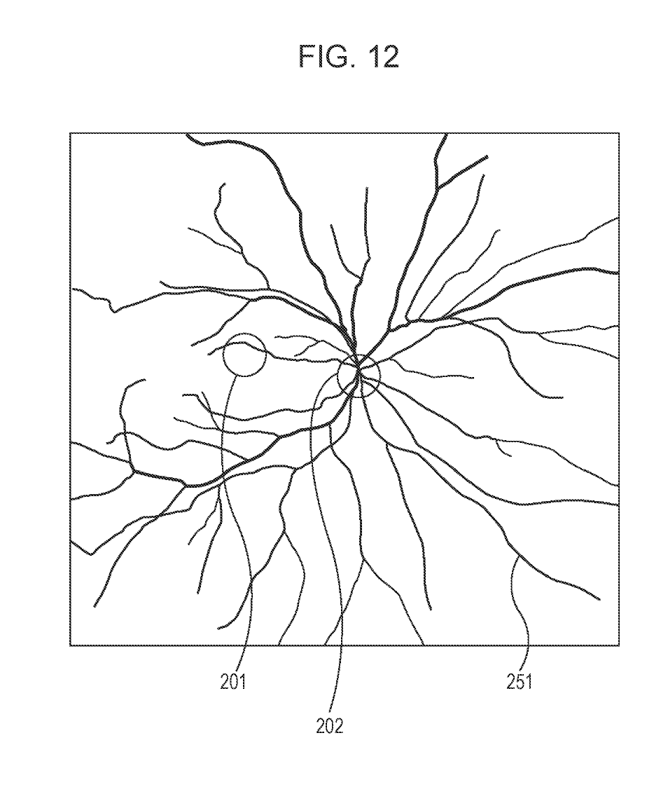

In order to generate an ocular fundus image with a wide field of view, it is necessary to photograph a wide area of an ocular fundus. Accordingly it is also necessary to move the fixation target which guides the eyepoint, across a wide range, for example, as illustrated in FIG. 9A or 9B. As a result of satisfying these necessities, a high-quality image with less noticeable borders is generated and provided, as illustrated in FIG. 12. FIG. 12 illustrates the exemplary wide-field ocular fundus image.

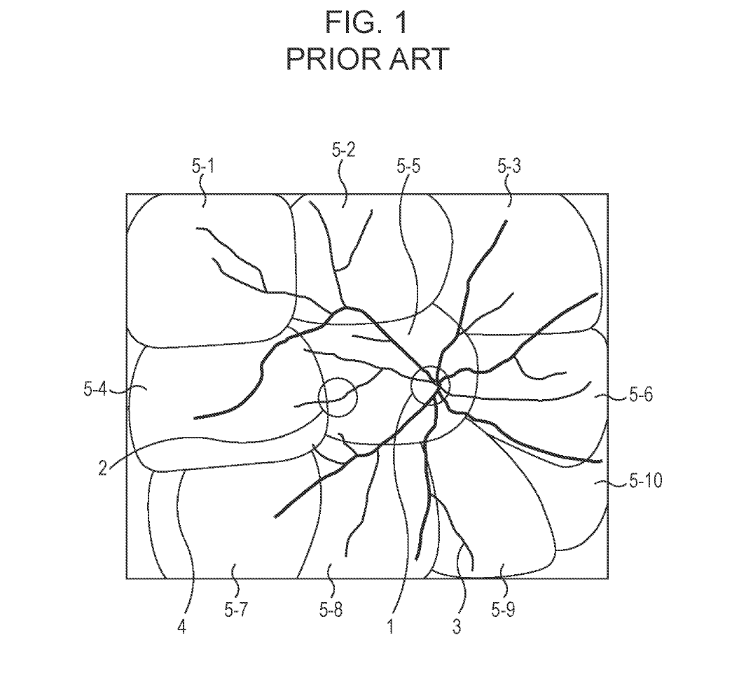

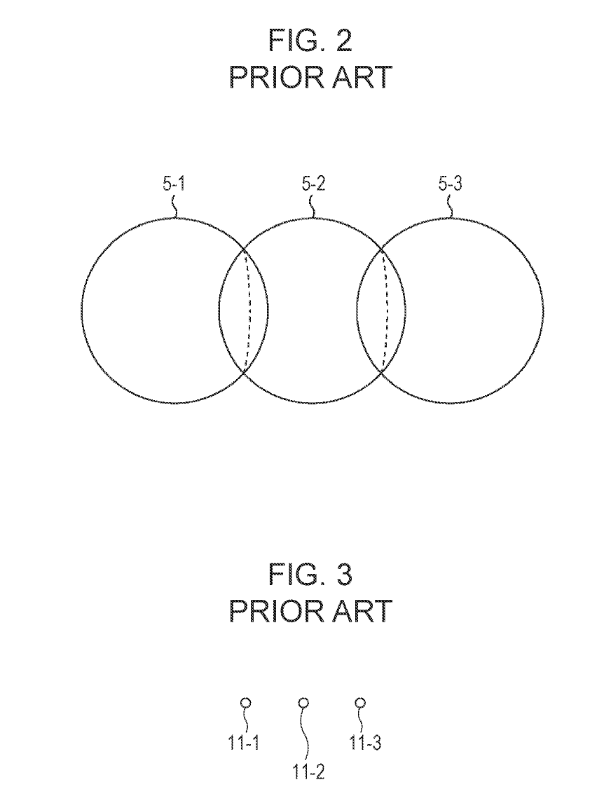

According to the technique illustrated in FIG. 1, a small number of images are pieced together in order to generate an image with a wide field of view. Therefore the borders between the adjacent images may become noticeable. In contrast, according to the first embodiment as in FIG. 12, a large number of images are synthesized for each pixel, so that a high-quality ocular fundus image with a wide field of view that has less noticeable borders is acquired.

FIG. 13 is an explanatory view of a method of synthesizing images. In the first embodiment, as illustrated in FIG. 13, the corresponding pixel values in a large number of sequential frame images are weighted and added, so that a high-quality image which has less noticeable borders is provided. In FIG. 13, a circular region encircled by a dotted line 281 corresponds to an image extracted from a single frame. A large number of block images are contained in this image.

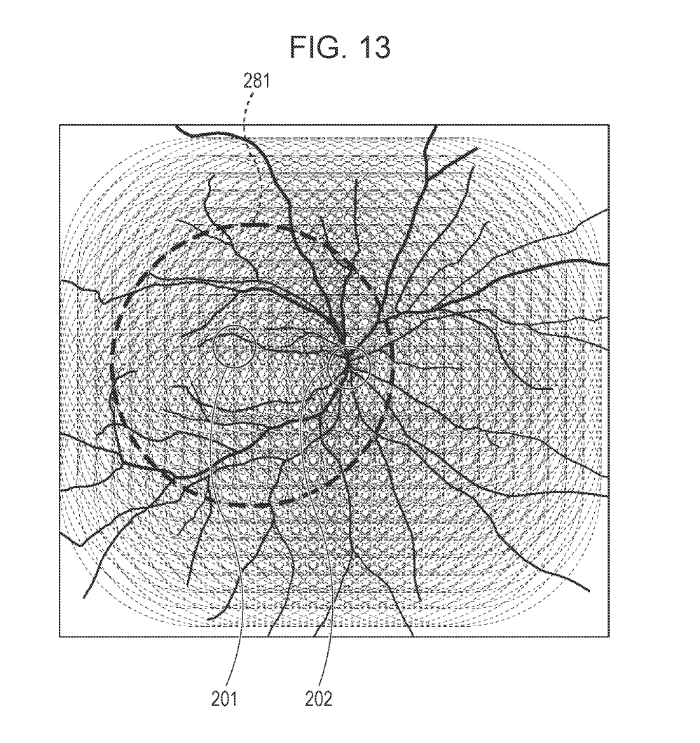

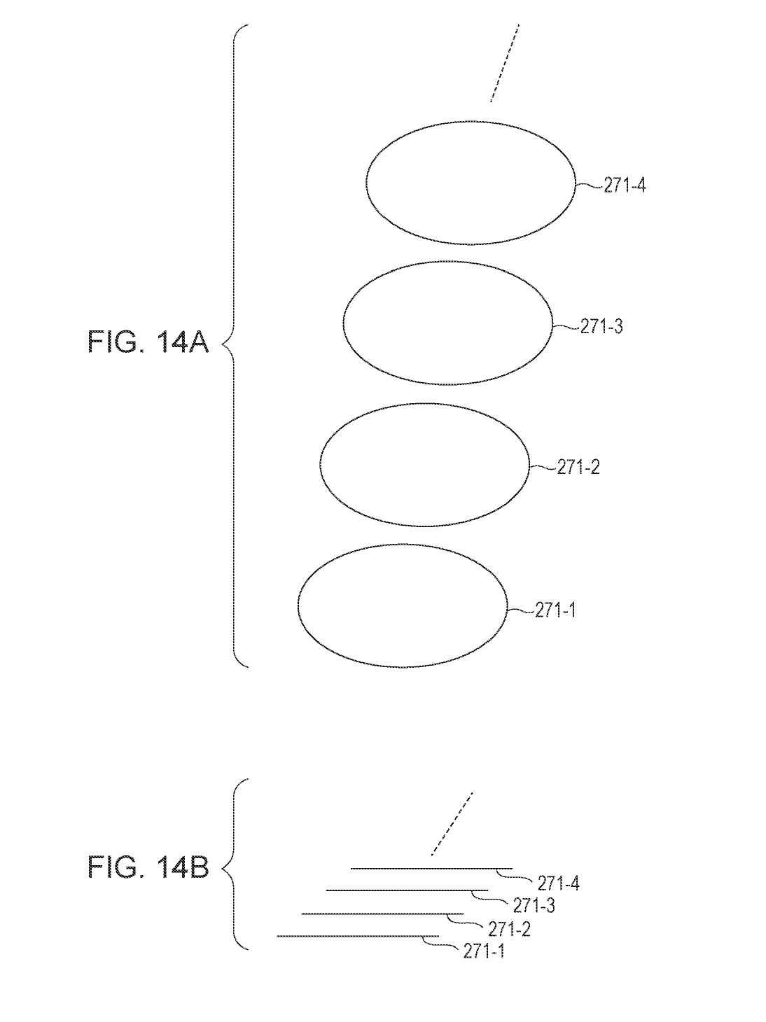

FIGS. 14A and 14B are explanatory, schematic views of the method of synthesizing frame images; FIG. 14A is a perspective view of the frame images and FIG. 14B is a side view of the frame images. In the first embodiment, as illustrated in FIG. 14A, a first image 271-1 to a fourth image 271-4 with a predetermined area (a circular region with a predetermined radius in the case of FIGS. 14A and 14B) are extracted from sequential frame images. Each of the images 271-1 to 271-4 corresponds to the image with the area encircled by the dotted line 281 in FIG. 13. In this case only the four images 271-1 to 271-4 are illustrated, but images of many more frame images are, in fact, extracted. For example, these images are extracted from sequential frame images which have been acquired while the fixation target 151 was continuously moving so as to trace the locus 152 in FIG. 9A or the locus 153 in FIG. 9B.

The respective areas contained in the first image 271-1 and the second image 271-2 are slightly shifted from each other. However since the images 271-i (i=1, 2, 3 and so on) are sequential frame images, the respective circular areas of the images 271-i, each of which is created by drawing a circle with the predetermined radius at the center of the photographic area, overlap one another by large amounts. As illustrated in FIG. 14B, corresponding parts are detected from the frame images, for example, through block matching, and the detected parts are weighted and added so as to overlay each other. Consequently the borders between the adjacent frame images in the resultant image become less noticeable, because the majority of the resultant image is made up of weighted and added pixels.

### Second Embodiment

### Acquisition of Super-Resolution Ocular Fundus Image

(Ocular Fundus Image with Super Resolution)

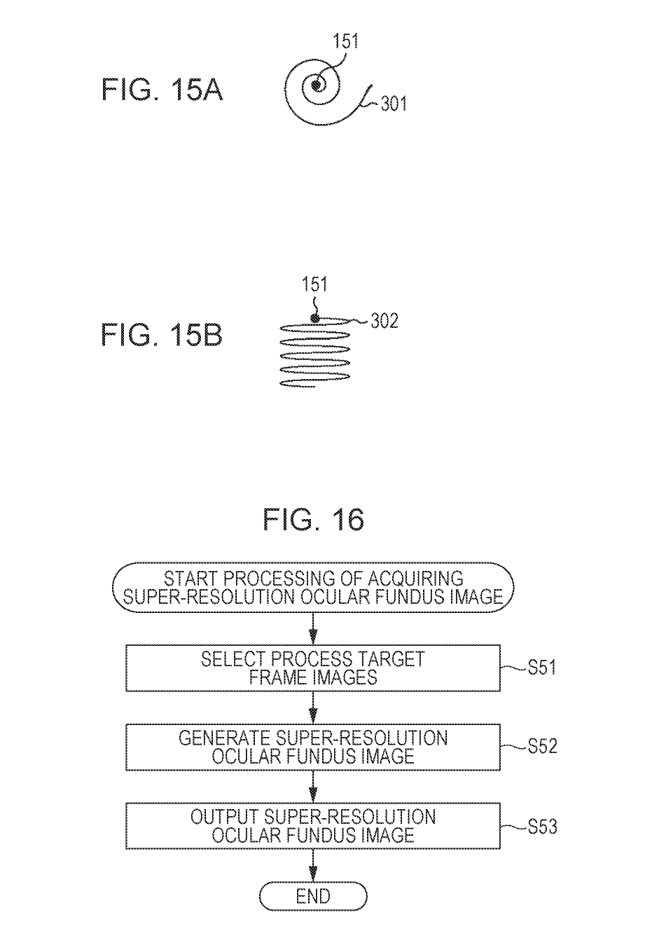

Next a description will be given regarding a case where information to be acquired is an ocular fundus image with a super resolution. It is known that applying the multiple frame super resolution technique results in the provision of images with great sharpness. In this case as for a positional relationship between one point in each ocular fundus image and a pixel of the image capturing element in the ocular fundus image acquisition section 31 which captures this point, it is necessary that this positional relationship differs in a smaller region than the spacing between the pixels, from one of the ocular fundus images to another one. It is desirable that the same point in the ocular fundus images have different positional relationships with the pixel, as described above, and wide-field information on the ocular fundus is not necessarily necessary. For this reason, for example, the fixation target that moves in a shorter range than the case of acquiring an ocular fundus image with a wide field of view (the case in FIGS. 9A and 9B) is used, as illustrated in FIGS. 15A and 15B.

FIGS. 15A and 15B are explanatory views of the movement of the fixation target when an ocular fundus image with a super resolution is acquired; FIG. 15A illustrates the exemplary fixation target 151 that moves from the inner side toward the outer side so as to trace a spiral locus 301, and FIG. 15B illustrates the exemplary fixation target 151 that moves so as to trace a sinusoidal locus 302. As is clear from FIGS. 15A and 9A, a region for the locus 301 in FIG. 15A is smaller than that for the locus 152 in FIG. 9A.

FIG. 16 is a flowchart of processing of acquiring a super-resolution ocular fundus image. Referring to FIG. 16, the process of acquiring a super-resolution ocular fundus image will be described.

At Step S51, the selection section 81 selects process target frame images from the frame images that make up a moving image received from the ocular fundus image acquisition section 31. This selection process may be performed as necessary, similar to the process at Step S1 in FIG. 11. At Step S52, the generation section 83 overlaps the process target frame images selected in the process at Step S51 while adjusting their relative position, thereby generating a super-resolution ocular fundus image. At Step S53, the output section 84 outputs the super-resolution ocular fundus image generated in the process at Step S52.

### (Another Configuration of Ocular Fundus Information Acquisition Section)

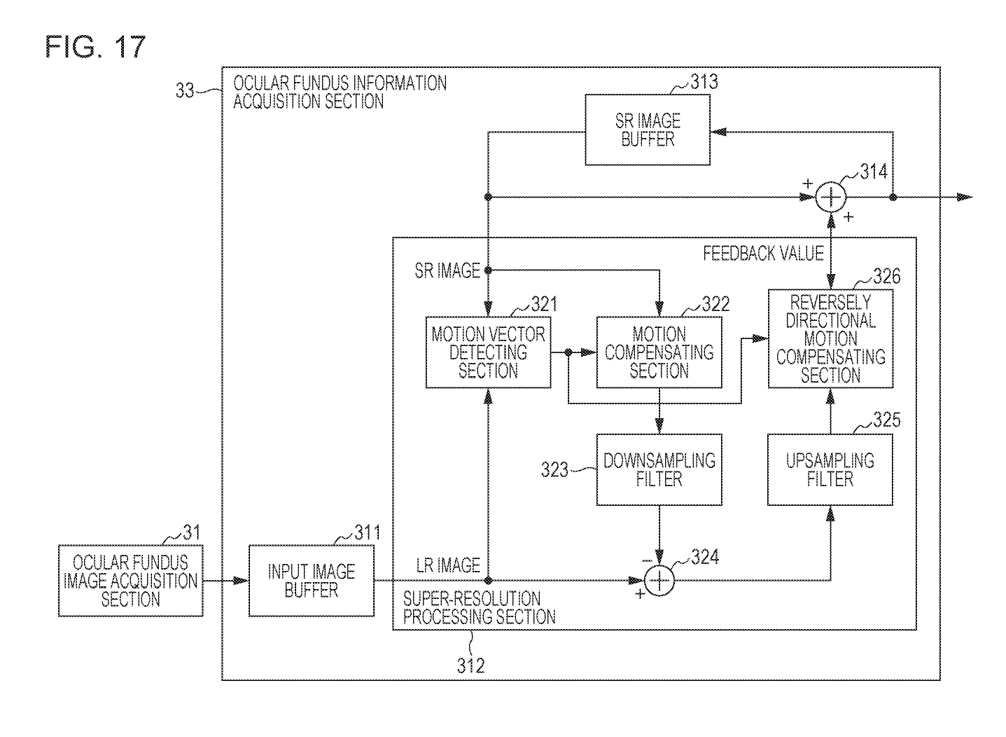

A description will be given of detail of the processing of acquiring a super-resolution ocular fundus image. For example, the ocular fundus information acquisition section 33 may be configured as in FIG. 17. FIG. 17 is a block diagram illustrating the exemplary functional configuration of the ocular fundus information acquisition section 33 when a super-resolution ocular fundus image is acquired.

The ocular fundus information acquisition section 33 generates a single high-quality ocular fundus image on the basis of a moving image of a ocular fundus, made up of multiple frame images, supplied from the ocular fundus image acquisition section 31, and then outputs the high-quality ocular fundus image.

As illustrated in FIG. 17, the ocular fundus information acquisition section 33 includes an input image buffer 311, a super-resolution processing section 312, a super-resolution (SR) image buffer 313, and a calculating section 314.

The input image buffer 311 has any given recording medium including, for example, a hard disk, a flash memory, and a random access memory (RAM). The input image buffer 311 retains the moving image supplied from the ocular fundus image acquisition section 31 as an input image. The input image buffer 311 then supplies the frame images making up the input image to the super-resolution processing section 312 at a preset timing, as low-resolution (LR) images.

### (Configuration of Super-Resolution Processing Section)

The super-resolution processing section 312 performs a super-resolution process, for example, which is the same as that performed by a super-resolution processor described in Japanese Unexamined Patent Application Publication No. 2009-093676. In more detail the super-resolution processing section 312 recursively repeats the super-resolution process. In this super-resolution process, both the LR image supplied from the input image buffer 311 and the SR image, generated in the past, supplied from the SR image buffer 313 are used to calculate a feedback value by which a new SR image is to be generated, and this feedback value is output. The super-resolution processing section 312 supplies the calculated feedback value to the calculating section 314, as a result of the super-resolution process.

The SR image buffer 313 has any given recording medium including, for example, a hard disk, a flash memory, and a RAM. In addition, the SR image buffer 313 retains the generated SR image, and supplies the SR image to the super-resolution processing section 312 or the calculating section 314 at a preset timing.

The calculating section 314 adds the feedback value supplied from the super-resolution processing section 312 to the SR image, generated in the past, supplied from the SR image buffer 313, thereby generating a new SR image. The calculating section 314 supplies the generated new SR image to the SR image buffer 313; the SR image buffer 313 retains it. This SR image will be used for a next super-resolution process (i.e. the generation of a new SR image). Furthermore, the calculating section 314 outputs the generated SR image to, for example, an external device.

As illustrated in FIG. 17, the super-resolution processing section 312 includes a motion vector detecting section 321, a motion compensating section 322, a downsampling filter 323, a calculating section 324, an upsampling filter 325, and a reversely directional motion compensating section 326.

The SR image read from the SR image buffer 313 is supplied to both the motion vector detecting section 321 and the motion compensating section 322. The LR image read from the input image buffer 311 is supplied to both the motion vector detecting section 321 and the calculating section 324.

The motion vector detecting section 321 detects a motion vector with reference to the SR image, on the basis of both the received SR image and LR image. The motion vector detecting section 321 then supplies the detected motion vector to both the motion compensating section 322 and the reversely directional motion compensating section 326.

The motion compensating section 322 subjects the SR image to motion compensation on the basis of the motion vector supplied from the motion vector detecting section 321. An image acquired as a result of the motion compensation is supplied to the downsampling filter 323. The location of a target object appearing in the image acquired as a result of the motion compensation is close to that in the LR image.

The downsampling filter 323 downsamples the image supplied from the motion compensating section 322, thereby generating an image that has the same resolution as the LR image. The downsampling filter 323 then supplies the generated image to the calculating section 324.

As described above, the motion vector is determined on the basis of both the SR image and the LR image, and the image that has been subjected to the motion compensation using this motion vector has the same resolution as the LR image. This processing is equivalent to that of simulating the captured ocular fundus image (LR image) on the basis of the SR image stored in the SR image buffer 313.

The calculating section 324 generates a differential image that indicates a difference between the LR image and the image simulated in the above manner, and supplies the generated differential image to the upsampling filter 325.

The upsampling filter 325 upsamples the differential image supplied from the calculating section 324, thereby generating an image that has the same resolution as the SR image. The upsampling filter 325 then outputs the generated image to the reversely directional motion compensating section 326.

The reversely directional motion compensating section 326 subjects the image supplied from the upsampling filter 325 to motion compensation in the reverse direction on the basis of the motion vector supplied from the motion vector detecting section 321. The feedback value that indicates an image acquired as a result of the motion compensation in the reverse direction is supplied to the calculating section 314. The location of a target object appearing in the image acquired as a result of the motion compensation in the reverse direction is close to that in the SR image stored in the SR image buffer 313.

The ocular fundus information acquisition section 33 subjects multiple frame images (LR images) stored in the input image buffer 311 to the above super-resolution process by using the super-resolution processing section 312. Consequently a single high-quality SR image is generated.

### (Processing of Generating Ocular Fundus Image)

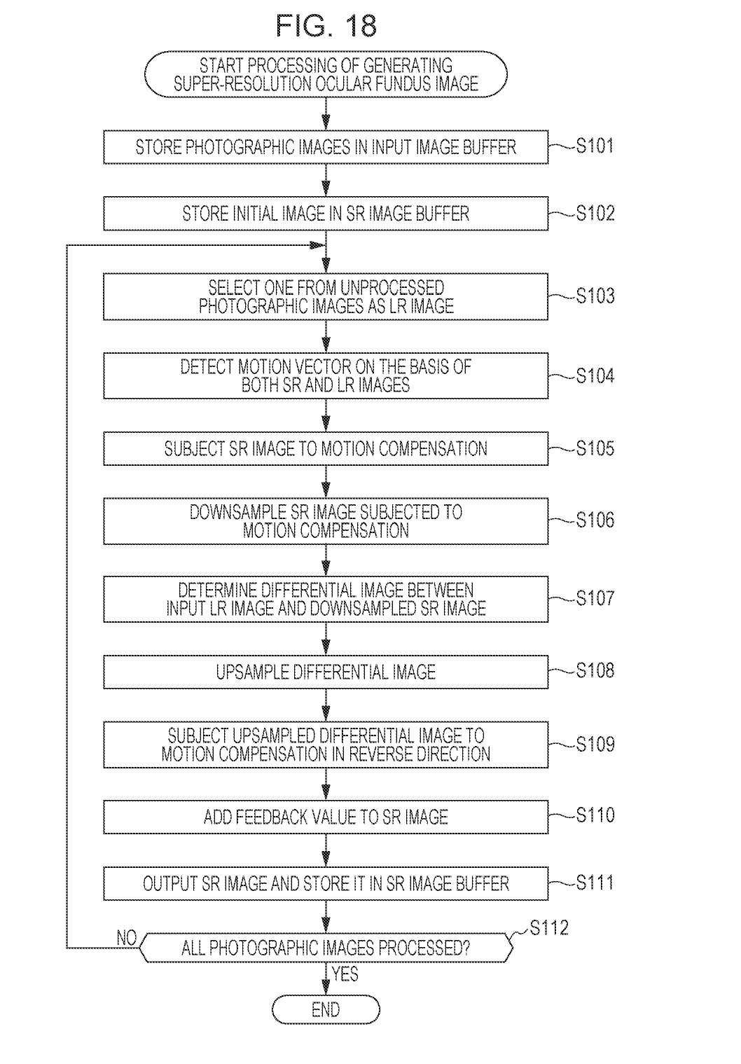

FIG. 18 is a flowchart of processing of generating a super-resolution ocular fundus image. Referring to the flowchart in FIG. 18, a description will be given of an exemplary process of generating a super-resolution ocular fundus image, which is performed by the ocular fundus information acquisition section 33. In the following example, the process of selecting the frame images is not performed.

At Step S101, the ocular fundus information acquisition section 33 stores, in the input image buffer 311, frame images making up a moving image acquired through the photography, as photographic images. At Step S102, the ocular fundus information acquisition section 33 generates a first SR image as an initial image by employing a predetermined method, and stores it in the SR image buffer 313. The ocular fundus information acquisition section 33 may generate the initial image, for example, by upsampling a first frame image (LR image) of the photographic images in such a way that the first frame image has the same resolution as the SR image.

At Step S103, the input image buffer 311 selects one from the unprocessed photographic images (LR images) retained therein, and supplies it to the super-resolution processing section 312. At Step S104, the motion vector detecting section 321 detects a motion vector on the basis of both the SR image and the LR image. At Step S105, the motion compensating section 322 subjects the SR image to the motion compensation by using the detected motion vector.

At Step S106, the downsampling filter 323 downsamples the SR image that has been subjected to the motion compensation in such a way that this SR image has the same resolution as the LR image. At Step S107, the calculating section 324 determines a differential image between the input LR image and the downsampled SR image.

At Step S108, the upsampling filter 325 upsamples the differential image. At Step S109, the reversely directional motion compensating section 326 subjects the upsampled differential image to the motion compensation in the reverse direction by using the motion vector detected in the process at Step S104.

At Step S110, the calculating section 314 adds the feedback value to the SR image, generated in the past, retained in the SR image buffer 313, the feedback value indicating the upsampled differential image which has been calculated in the process at Step S109. The ocular fundus information acquisition section 33 outputs the newly generated SR image at Step S111, and stores it in the SR image buffer 313.

At Step S112, the input image buffer 311 determines whether or not all the photographic images (LR images) have been processed. When it is determined that at least one unprocessed photographic image (LR images) is present (“NO” at Step S112), the ocular fundus information acquisition section 33 returns the current processing to the process at Step S103. Then the ocular fundus information acquisition section 33 selects a new photographic image as a process target, and subjects this process target to the subsequent processes again.

When it is determined that all the photographic images making up the moving image supplied from the ocular fundus image acquisition section 31 have been processed and a single high-quality ocular fundus image has been acquired (“YES” at Step S112), the input image buffer 311 terminates the processing of generating the super-resolution ocular fundus image.

Through the above processing, a high-quality ocular fundus image is acquired by the ocular fundus information acquisition section 33.

The above super-resolution process may be performed for each desired unit. For example, the photographic image may be entirely processed at one time. Alternatively the photographic image may be separated into multiple partial images, or macro blocks, with a preset area, and these macro blocks may be processed individually.

### Third Embodiment

### Acquisition of 3D Shape

### (Acquisition of 3D Shape)

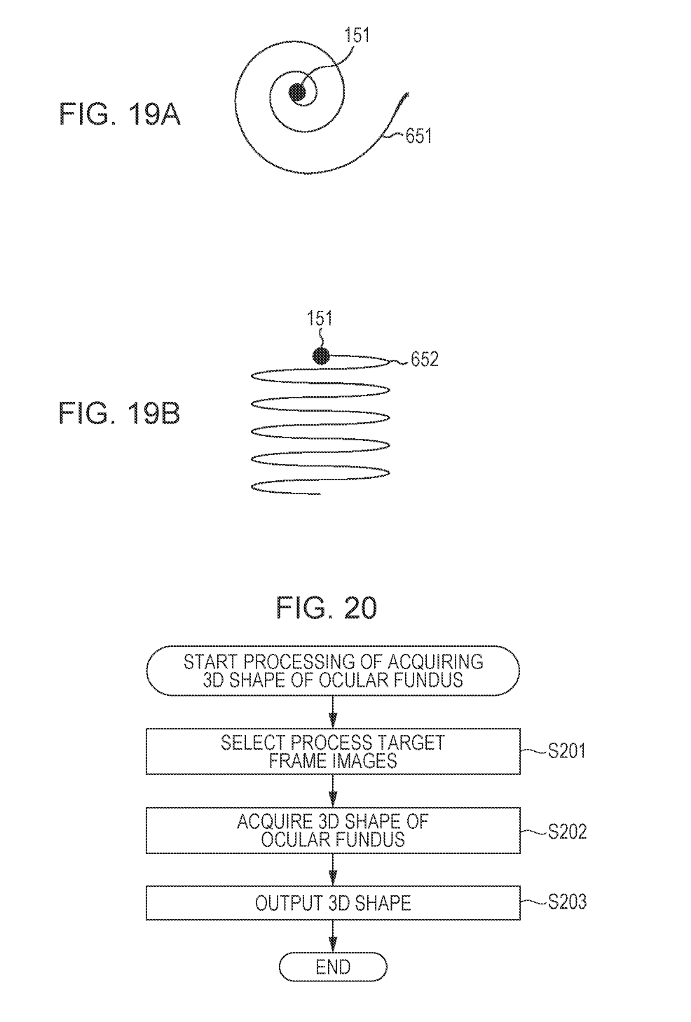

Next a description will be given regarding a case where information to be acquired is a 3D shape of the ocular fundus. In order to acquire this 3D shape, it is necessary to photograph the ocular fundus at somewhat different angles which the camera forms with the ocular fundus. Therefore, as illustrated in FIGS. 19A and 19B, for example, the fixation target is forced to move in an intermediate range between those used to acquire the wide-field and super-resolution ocular fundus images. FIGS. 19A and 19B are explanatory views of the movement of the fixation target. In comparison with FIGS. 9A, 9B, 15A and 15B, a region for the spiral locus 651 in FIG. 19A is smaller than that for the spiral locus 152 in FIG. 9A drawn to acquire the wide-field ocular fundus image and larger than that for the spiral locus 301 in FIG. 15A drawn to acquire the super-resolution ocular fundus image. Likewise a region for the spiral locus 652 in FIG. 19B is smaller than that for the spiral locus 153 in FIG. 9B and larger than that for the spiral locus 302 in FIG. 15B.

FIG. 20 is a flowchart of processing of acquiring a 3D shape of the ocular fundus. Referring to FIG. 20, a description will be given of processing of acquiring the 3D shape of the ocular fundus, which is performed by the ocular fundus information acquisition section 33.

At Step S201, the selection section 81 selects process target frame images from the image frames making up an input moving image. This selection may be made as necessary, similar to the process at Step S1 in FIG. 11. At Step S202, the acquisition section 82 acquires a 3D shape of the ocular fundus on the basis of a positional relationship among the respective ocular fundi in the process target frame images selected in the process at Step S201.

In order to acquire a 3D shape, for example, the structure from motion (SFM) technique may be employed. In the SFM technique, the moving image of a certain target is captured by a camera while the camera is being moved, and the shape of the certain target is estimated from the captured moving image. The Tomasi-Kanade factorization is a typical method that implements the SFM technique. In this method, p pairs of corresponding points are acquired from an F number of time-series images captured, and a 2F×P matrix is created from the group of the corresponding points. This matrix has a rank of three or less, and is therefore decomposed into respective matrixes expressing the 3D locations of the feature points and the locations of the camera.

In the third embodiment, the moving image of the ocular fundus is not captured by the moving camera. Instead it is captured while the direction in which the subject's eye 41, substantially regarded as a rigid body, faces is being changed. As a result it is possible to acquire an ocular fundus image which is equivalent to that acquired under the condition that the subject's eye 41 faces in a fixed direction and the camera is moving. For this reason the SFM is applicable to the third embodiment. Various specific methods that employ the SFM technique have been proposed so far; exemplary literatures describing the methods are listed below.
C. Tomasi and T. Kanade, Shape and Motion from Image Streams under Orthography: a Factorization Method, International Journal of Computer Vision, 9:2, 137-154, 1992
C. J. Poelman and T. Kanade, A Paraperspective Factorization Method for Shape and Motion Recovery, IEEE Transactions on Pattern Analysis and Machine Intelligence, Vol. 19, No. 3, 1997

At Step S203, the output section 84 outputs the 3D shape of the ocular fundus which has been acquired in the process at Step S202.

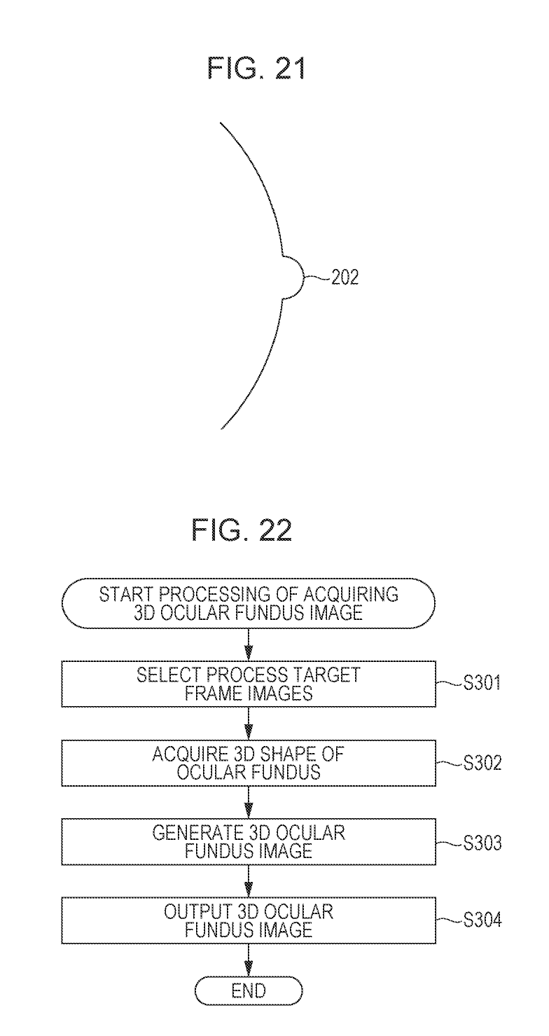

Through the above processing, the 3D shape of the ocular fundus is acquired, for example, as illustrated in FIG. 21. FIG. 21 illustrates a cross section of an exemplary 3D shape of the ocular fundus. In the example of FIG. 21, the cross section of the ocular fundus in the vicinity of the optic papilla 202 is illustrated. The shape of the optic papilla 202 is effective for, for example, the diagnosis of the glaucoma.

### Fourth Embodiment

### Acquisition of 3D Ocular Fundus Image

### (3D Ocular Fundus Image)

Next a description will be given regarding a case where information to be acquired is a 3D ocular fundus image. The movement of the fixation target in this case is the same as that when the 3D shape of the ocular fundus is acquired (FIGS. 19A and 19B).

FIG. 22 is a flowchart of processing of acquiring a 3D ocular fundus image. Referring to FIG. 22, a description will be given below of processing of acquiring a 3D ocular fundus image which is performed by the ocular fundus information acquisition section 33.

At Step S301, the selection section 81 selects process target frame images from the frame images making up an input moving image. This selection may be made as necessary, similar to the selection process at Step S1 in FIG. 11. At Step S302, the acquisition section 82 acquires a 3D shape of the ocular fundus on the basis of a positional relationship among the respective ocular fundi in the process target frame images selected in the process at Step S301.

At Step S303, the generation section 83 maps the ocular fundus image onto the 3D shape acquired in the process at Step S302, in accordance with information on the corresponding positions of an ocular fundus that has already been determined, thereby generating a 3D ocular fundus image. In this case the mapped ocular fundus image may be an arbitrary one of the selected frame images. Alternatively if the ocular fundus appears in multiple frame images at the same position, an ocular fundus image generated by weighting and adding these frame images may be used. At Step S304, the output section 84 outputs the 3D ocular fundus image generated in the process at Step S303.

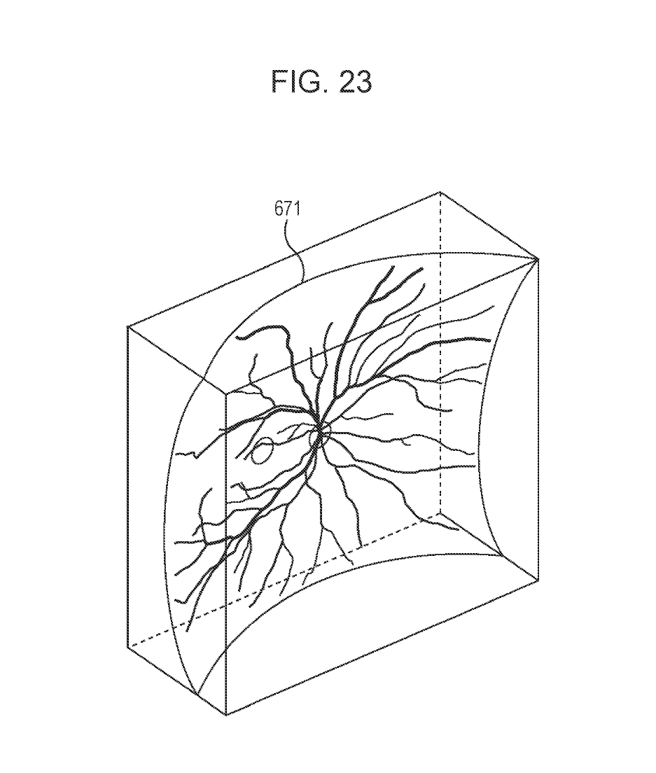

FIG. 23 illustrates an exemplary 3D ocular fundus image. The image in FIG. 23 is an example of the 3D ocular fundus image that is output in the process at Step S304. In FIG. 23 the ocular fundus image is displayed on the curved surface 671.

As described above, the ocular fundus information acquisition section 33 selects frame images at the first step, regardless of which information to be acquired. However in the case where the external fixation target is used, all the frame images may be selected. Even in the case where the internal fixation target is used, all the frame images may also be selected as long as the moving image acquired by the ocular fundus image acquisition section 31 is an infrared image and has been captured through an infrared light transmission filter in order to reduce the influence of the fixation target, as described above.

In the case where the internal fixation target is used and a moving image to be acquired is a visible light image, the moving image is unable to be captured through an infrared light transmission filter (visible light cut filter). This is because visible light to be photographed does not reach the image capturing element. In this case it is necessary to blink the internal fixation target and to select only image frames that have been captured while the fixation target is not lighted, as described with reference to FIGS. 7A and 7B. This enables the moving image to be acquired without being affected by the light of the fixation target. The determination whether or not a frame image has been captured during the non-lighting period of the fixation target may be made from the control information on the fixation target. Alternatively this determination may be made by image processing referring to captured images. In other words image frames that do not contain the fixation target may be detected and selected.

### Fifth Embodiment

### Configuration with Ocular Fundus Image Provision Section

### (Another Configuration of Ocular Fundus Information Acquisition Device)

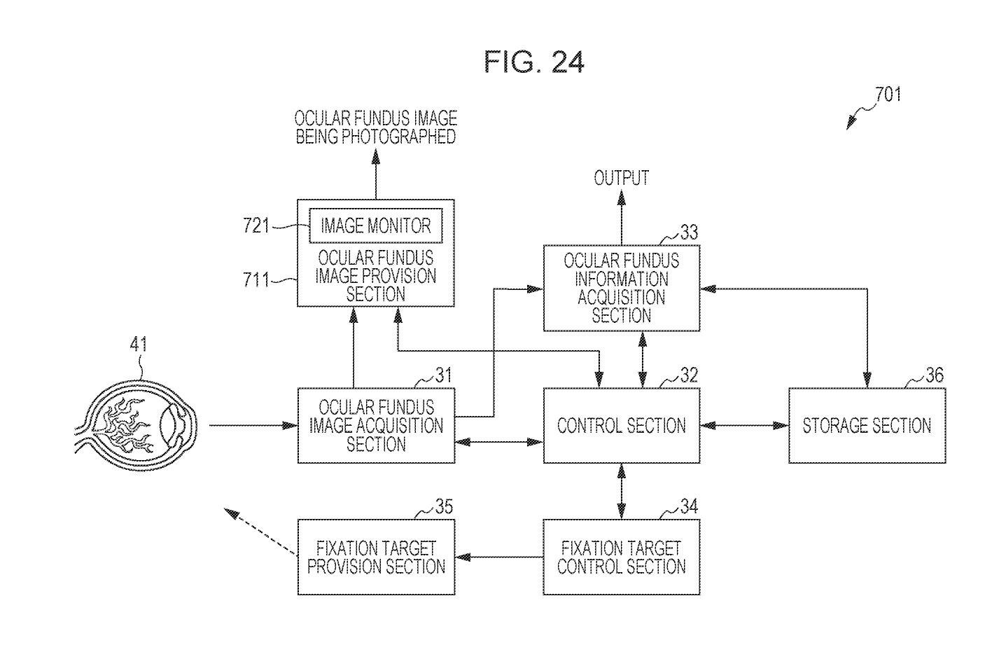

FIG. 24 is a block diagram illustrating an exemplary configuration of an ocular fundus information acquisition device 701. In FIG. 24, an ocular fundus image acquisition device 701 having an ocular fundus image provision section 711 is illustrated. This configuration is provided with, as an additional component, the ocular fundus image provision section 711 which provides an image of the ocular fundus being photographed. In other respects this configuration is like that in FIG. 4.

The ocular fundus information acquisition device 701 in FIG. 24 captures a moving image by using the ocular fundus image acquisition section 31, and displays this moving image on an image monitor 721 in the ocular fundus image provision section 711. This enables the photographer to perform the photographing operation while monitoring the captured image on the image monitor 721.

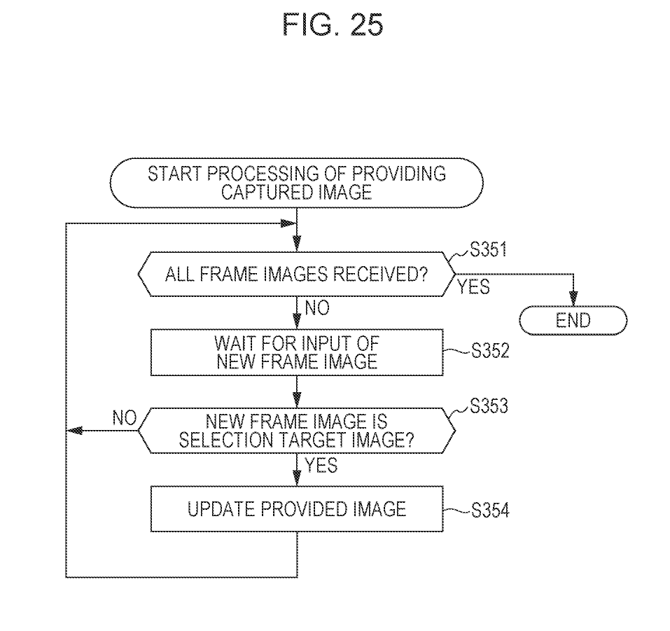

In the case where a visible light moving image is captured using the internal fixation target, the image acquired by the ocular fundus image acquisition section 31 may be entirely and directly displayed on the image monitor 721 in the ocular fundus image provision section 711. If the internal fixation target blinks, an image of the blinking internal fixation target is displayed on the image monitor 721. This may cause the photographer to feel inconvenienced. Accordingly in order to reduce this inconvenience, the target frame images may be selected. Then only the selected images may be provided to the image monitor 721, and the image monitor 721 may update its displayed image with these images, as in FIG. 25.

FIG. 25 is a flowchart illustrating processing of providing a captured image. At Step S351, the selection section 81 determines whether to have received all frame images. When all the frame images have already been received (“YES” at Step S351), the ocular fundus information acquisition device 701 terminates this processing. When all the frame images have not yet been received (“NO” at Step S351), the selection section 81 waits for the input of a new frame image at Step S352.

Upon receiving a new frame image, the selection section 81 determines whether or not the new frame image is a selection target image at Step S353. Here the selection target image is a frame image captured while the fixation target is not lighted, for example, as described with reference to FIGS. 7A and 7B. When the new frame image is not the selection target image (“NO” at Step S353), the ocular fundus information acquisition device 701 returns the current processing to the process at Step S351, and repeats the subsequent processes.

When the new frame image is the selection target image (“YES” at Step S353), the selection section 81 updates a provided image at Step S354. In more detail the image that has been provided by the image monitor 721 is updated to the new frame image. In other words the selection target frame image that has been previously received (the last frame image that has been captured during the non-lighting period of the fixation target) is not updated, or is continuously provided, until a new selection target frame image is received. Since a frame image that has been captured immediately before the lighting of the fixation target is continuously provided, there is no possibility that the photographer views an unwanted image on the image monitor 721. This eliminates a risk of causing the photographer to feel inconvenienced. After that the ocular fundus information acquisition device 701 returns the current processing to the process at Step S351, and repeats the subsequent processes.

### Sixth Embodiment

### Acquisition of Moving Image with Infrared Light

(Acquisition of Moving Image with Infrared Light and Still Image with Visible Light)

In order to acquire a 3D visible light ocular fundus image, the ocular fundus image acquisition section 31 may first acquire a moving image with infrared light, and then acquire a still image with visible light. By mapping the visible light still image onto the 3D shape of the ocular fundus which is acquired from the infrared light moving image while adjusting the position of the visible light still image with respect to the infrared light moving image, the 3D visible light ocular fundus image is acquired.

When the capturing of the still image with visible light is performed first, it is necessary for a mydriatic agent to be applied to the subject's eye 41 prior to the capturing with infrared light, in order to prevent the subject's eye 41 from causing the pupillary constriction. In contrast when the moving image is first captured with infrared light and the still image is then captured with visible light, the visible light shines on the subject's eye 41 only when the still image is captured. This eliminates the necessity to apply a mydriatic agent, similar to a case of using a non-mydriatic fundus camera, thereby reducing the inconvenience for the subject.

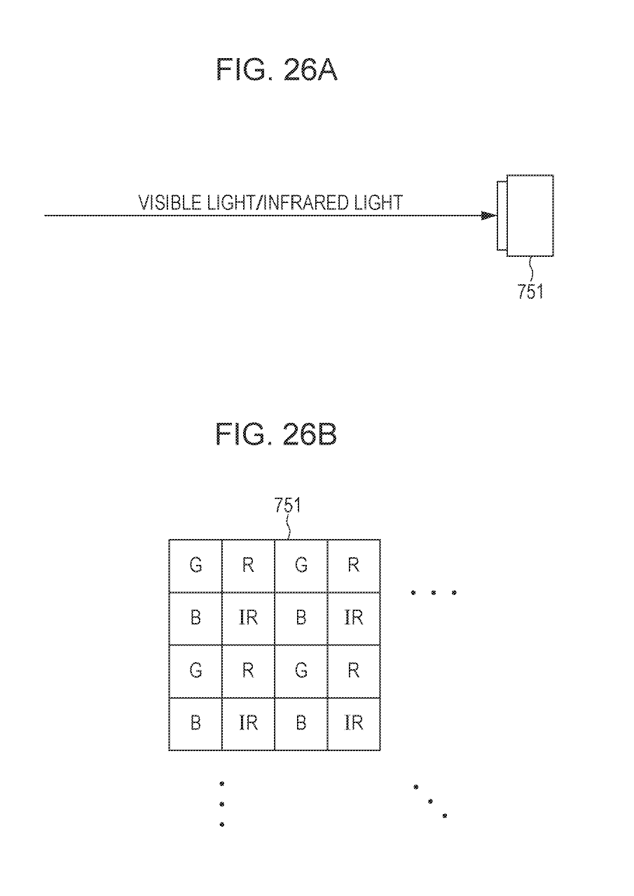

Both the infrared light moving image and the visible light still image may be captured by the fixation target provision section 35A configured as in FIG. 6. In this case a configuration as illustrated in FIGS. 26A and 26B may be used to capture the images. FIGS. 26A and 26B are explanatory views of an image capturing element that captures a moving image with infrared light and a still image with visible light. An image capturing element 751 in FIG. 26A receives both infrared light and visible light. As illustrated in FIG. 26B, the image capturing element 751 has light receiving parts arranged in a matrix fashion; out of these light receiving parts, some denoted by letters R, G and B receive visible light and the other denoted by letters IR receive infrared light. For the pixels in the image capturing element 751, color filters that transmit visible light beams such as red, green and blue and IR filters that transmit infrared light beams are used.

The infrared light moving image is acquired through the pixels provided with the IR filters, and the visible light still image is acquired through pixels provided with the R, G and B filters. In the sixth embodiment no change in the photographic light path is necessary.

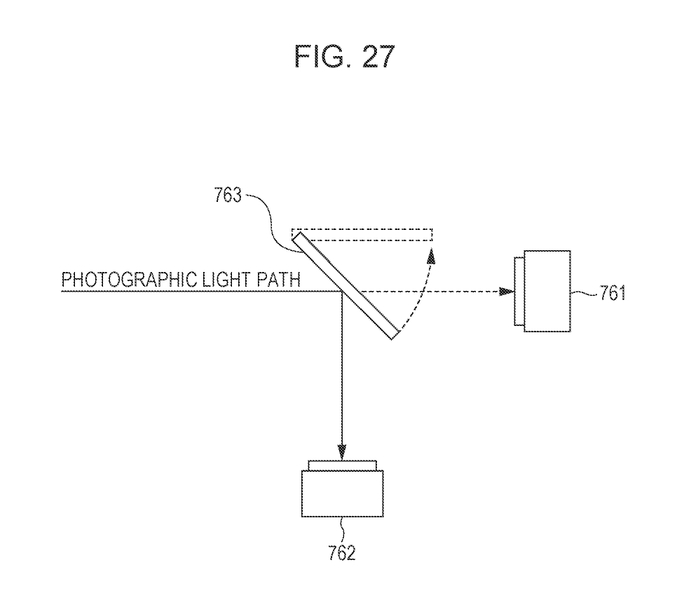

In order to acquire both the infrared light moving image and the visible light still image, a configuration illustrated in FIG. 27 may also be used as a modification of the sixth embodiment. FIG. 27 is an explanatory view of a method of capturing a moving image with infrared light and a still image with visible light. In this modification both a visible light image capturing element 761 that receives visible light and an infrared light image capturing element 762 that receives infrared light are prepared. In addition a rotatable mirror 763 is disposed in the photographic light path.

Before the visible light image capturing element 761 receives visible light from the subject's eye 41, the rotatable mirror 763 rotates so as to be placed at a site represented by a dotted line in FIG. 27. As a result the visible light enters only the visible light image capturing element 761. Before the infrared light image capturing element 762 receives infrared light from the subject's eye 41, the rotatable mirror 763 rotates so as to be placed at a site represented by a solid line in FIG. 27. As a result the infrared light enters only the infrared light image capturing element 762 after being reflected by the rotatable mirror 763.

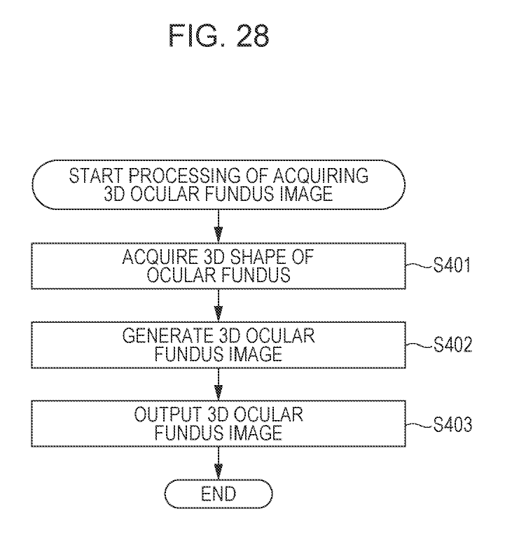

A description will be given of processing of acquiring a 3D visible light ocular fundus image by mapping a visible light still image onto a 3D shape of the ocular fundus which is acquired from an infrared light moving image while adjusting the position of the visible light still image with respect to the infrared light moving image, with reference to FIG. 28. FIG. 28 is a flowchart illustrating the processing of acquiring a 3D ocular fundus image.

At Step S401, the acquisition section 82 acquires a 3D shape of the ocular fundus on the basis of a positional relationship among the respective ocular fundi in frame images that make up an infrared light moving image received from the ocular fundus image acquisition section 31. At Step S402, the generation section 83 maps the visible light still image onto the 3D shape acquired in the process at Step S401 while adjusting the position of the visible light still image with respect to the 3D shape, thereby generating a 3D ocular fundus image. At Step S403, the output section 84 outputs the 3D ocular fundus image generated in the process at Step S402.

As described above, the embodiments of the present technology simply and easily provide a high-quality ocular fundus image with a wide field of view, an ocular fundus image with a super resolution, a 3D shape of the ocular fundus, and a 3D ocular fundus image, without causing a photographer to feel inconvenienced. In addition the embodiments of the present technology successfully reduce the inconvenience for a subject which would occur when a 3D visible light ocular fundus image is acquired.

### [Application of Present Technology to Program]

The series of processes, as described above, may be performed by either hardware or software.

When the series of processes are performed by software, a program configuring this software is installed, via a network or a recording medium, in a computer built into dedicated hardware or a general-purpose personal computer that is capable of performing various functions after the installation of corresponding programs.

The recording medium that stores the above program may be independent of the main body of the device and be a removable medium to be distributed to provide a user with the program. Examples of the removable medium include, but are not limited to, a magnetic disk such as a flexible disk, an optical disc such as a compact disk-read only memory (CD-ROM) or a digital video disc (DVD), and a semiconductor memory. Alternatively the recording medium may be the storage section 36 configured with a flash ROM or a hard disk that stores the program and is to be provided to a user while being built into the main body of the device.

The program to be executed by a computer may sequentially perform the processes in order of the description herein or perform some of the processes in parallel. Moreover the program may perform the processes at an appropriate timing, for example, when the program is called.

An embodiment of the present technology is not limited to the above embodiments, and various modifications and variations are possible without departing the spirit of the present technology.

An exemplary configuration in an embodiment of the present technology may be a cloud computing in which a single function is shared by a plurality of devices via a network or fulfilled by their cooperation.

The process at the steps in each flowchart described above may be performed by a single device or performed separately by a plurality of devices.

If one of the steps contains a plurality of processes, these processes may be performed by a single device or performed separately by a plurality of devices.

### [Another Configuration]

The present technology may also have the following configuration.

(1) An ocular fundus information acquisition device including: a fixation target provision section configured to provide a continuously moving fixation target; an ocular fundus image acquisition section configured to acquire an image of an ocular fundus in a subject's eye while the subject is closely watching the continuously moving fixation target; and an ocular fundus information acquisition section configured to acquire ocular fundus information from the acquired ocular fundus image.
(2) The ocular fundus information acquisition device according to (1) wherein the ocular fundus image acquisition section acquires a moving image of the ocular fundus.
(3) The ocular fundus information acquisition device according to (1) or (2) wherein the fixation target provision section provides a blinking internal fixation target.
(4) The ocular fundus information acquisition device according to (3) wherein the ocular fundus information acquisition section selects, as a target image, a frame image in the moving image which has been acquired during a period in which the fixation target is not lighted, and the ocular fundus information is acquired from the selected target image.
(5) The ocular fundus information acquisition device according to one of (1) to (4), further including an ocular fundus image provision section configured to provide the image of the ocular fundus in the subject's eye which has been acquired while the subject is closely watching the continuously moving fixation target.
(6) The ocular fundus information acquisition device according to (5) wherein the ocular fundus image provision section provides the ocular fundus image during a period in which the fixation target is not lighted, the ocular fundus image being the frame image in the moving image, and provides the ocular fundus image during a period in which the fixation target is lighted, the ocular fundus image being the frame image in the moving image which has been acquired during the period in which the fixation target is not lighted.
(7) The ocular fundus information acquisition device according to one of (1) to (6) wherein the ocular fundus information acquisition section acquires the ocular fundus image with a wide field of view.
(8) The ocular fundus information acquisition device according to one of (1) to (6) wherein the ocular fundus information acquisition section acquires the ocular fundus image with super resolution.
(9) The ocular fundus information acquisition device according to one of (1) to (6) wherein the ocular fundus information acquisition section acquires a 3D shape of the ocular fundus.
(10) The ocular fundus information acquisition device according to one of (1) to (6) wherein the ocular fundus information acquisition section acquires a 3D ocular fundus image.
(11) The ocular fundus information acquisition device according to one of (1) to (6) wherein the ocular fundus image acquisition section acquires the moving image of the ocular fundus with infrared light and a still image of the ocular fundus with visible light, and the ocular fundus information acquisition section acquires a 3D shape of the ocular fundus from the infrared light moving image of the ocular fundus, and acquires a visible light 3D ocular fundus image by mapping the visible light still image onto the 3D shape while adjusting a location of the visible light still image with respect to the 3D shape.
(12) A method of acquiring ocular fundus information, including: providing a continuously moving fixation target; acquiring an image of an ocular fundus in a subject's eye while the subject is closely watching the continuously moving fixation target; and acquiring ocular fundus information from the acquired ocular fundus image.
(13) A program allowing a computer to perform processing including: providing a continuously moving fixation target; acquiring an image of an ocular fundus in a subject's eye while the subject is closely watching the continuously moving fixation target; and acquiring ocular fundus information from the acquired ocular fundus image.

It should be understood by those skilled in the art that various modifications, combinations, sub-combinations and alterations may occur depending on design requirements and other factors insofar as they are within the scope of the appended claims or the equivalents thereof.

What is claimed is:

1. An ocular fundus information acquisition device comprising:
a fixation target provision section configured to provide a continuously moving fixation target;
an ocular fundus image acquisition section configured to acquire an image of an ocular fundus in a subject's eye while the subject is closely watching the continuously moving fixation target; and
an ocular fundus information acquisition section configured to acquire ocular fundus information from the acquired ocular fundus image.

2. The ocular fundus information acquisition device according to claim 1, wherein
the ocular fundus image acquisition section acquires a moving image of the ocular fundus.

3. The ocular fundus information acquisition device according to claim 2, wherein
the fixation target provision section provides a blinking internal fixation target.

4. The ocular fundus information acquisition device according to claim 3, wherein
the ocular fundus information acquisition section selects, as a target image, a frame image in the moving image which has been acquired during a period in which the fixation target is not lighted, and the ocular fundus information is acquired from the selected target image.

5. The ocular fundus information acquisition device according to claim 4, further comprising:
an ocular fundus image provision section configured to provide the image of the ocular fundus in the subject's eye which has been acquired while the subject is closely watching the continuously moving fixation target.

6. The ocular fundus information acquisition device according to claim 5, wherein
the ocular fundus image provision section provides the ocular fundus image during a period in which the fixation target is not lighted, the ocular fundus image being the frame image in the moving image, and provides the ocular fundus image during a period in which the fixation target is lighted, the ocular fundus image being the frame image in the moving image which has been acquired during the period in which the fixation target is not lighted.

7. The ocular fundus information acquisition device according to claim 6, wherein
the ocular fundus information acquisition section acquires the ocular fundus image with a wide field of view.

8. The ocular fundus information acquisition device according to claim 6, wherein
the ocular fundus information acquisition section acquires the ocular fundus image with super resolution.

9. The ocular fundus information acquisition device according to claim 6, wherein
the ocular fundus information acquisition section acquires a 3D shape of the ocular fundus.

10. The ocular fundus information acquisition device according to claim 6, wherein
the ocular fundus information acquisition section acquires a 3D ocular fundus image.

11. The ocular fundus information acquisition device according to claim 10, wherein
the ocular fundus image acquisition section acquires the moving image of the ocular fundus with infrared light and a still image of the ocular fundus with visible light, and
the ocular fundus information acquisition section acquires a 3D shape of the ocular fundus from the infrared light moving image of the ocular fundus, and acquires a visible light 3D ocular fundus image by mapping the visible light still image onto the 3D shape while adjusting a location of the visible light still image with respect to the 3D shape.

12. A method of acquiring ocular fundus information, comprising:
providing a continuously moving fixation target;
acquiring an image of an ocular fundus in a subject's eye while the subject is closely watching the continuously moving fixation target; and
acquiring ocular fundus information from the acquired ocular fundus image.

13. A program allowing a computer to perform processing comprising:
providing a continuously moving fixation target;
acquiring an image of an ocular fundus in a subject's eye while the subject is closely watching the continuously moving fixation target; and
acquiring ocular fundus information from the acquired ocular fundus image.
